(19) United States
(12) Patent Application Publication
(10) Pub. No.: US 2025/0160687 A1
(43) Pub. Date: May 22, 2025

(54) CUEING DEVICE ALGORITHM

(71) Applicant: Cue2Walk International B.V., AL DEN HAAG (NL)

(72) Inventors: Alexander Leopold Marie MINNOYE, AL DEN HAAG (NL); Floor WAARDENBURG, AL DEN HAAG (NL); Martijn VAN DER ENT, AL DEN HAAG (NL)

(21) Appl. No.: 18/840,519

(22) PCT Filed: Feb. 23, 2023
(86) PCT No.: PCT/EP2023/054583

§ 371 Date: Aug. 22, 2024

(30) Foreign Application Priority Data
Feb. 24, 2022 (NL) 2031060
Feb. 24, 2022 (NL) 2031061
Feb. 24, 2022 (NL) 2031062

(51) Int. Cl. A61B 5/11; A61B 5/00; G16H 50/20

(52) U.S. Cl. CPC A61B 5/112; A61B 5/6831; G16H 50/20; A61B 2562/0219

(57) ABSTRACT

A cueing device is provided for producing a cue in assisting a user during a motion. The cueing device comprises a control unit operatively connected with a motion sensor and a cueing unit. The control unit is programmed with multiple algorithms which process the motion data from the motion sensor and providing multiple outcomes in determining a gait irregularity during the motion. A cue is only produced in case outcomes of at least two algorithms positively indicate for a gait irregularity. Cue production should be minimised to situations in which a cue is really necessary and desired by the user. Different situations and personal specifics can be better distinguished by using the at least two outcomes of the at least two gait irregularity algorithms. Unnecessary cue production can be reduced which prevents habituation and increases the effectiveness of the cueing device.

### FIELD OF THE DISCLOSURE

According to a first aspect of the invention, the invention relates to a cueing device, in particular a Parkinson cueing device comprising a control unit which is programmed by an algorithm providing an outcome to determine a gait irregularity during a movement of the user.

### BACKGROUND

Gait irregularities, such as freeze of gait (FoG) or festination, can occur in people who suffer from neurological diseases such as Parkinson's disease, cerebral palsy, and multiple sclerosis. Particularly for people who suffer from Parkinson's disease, gait irregularity typically occurs when walking and is reflected as a brief unexpected inability to walk which might cause a fall.

U.S. Ser. No. 10/251,611 discloses a wearable apparatus which automatically activates a cue. The apparatus has a housing that contains electronics including at least one processor and a motion sensor. The electronics include a power circuit utilising a battery for power storage and peripherals, such as a power switch, an indicator light, and the like. When turned on, the motion sensor of the apparatus starts to detect movements and outputs motion data to the processor. The processor uses a gait analysis algorithm to differentiate between normal walking and a gait irregularity such as a freeze of gait (FoG). If a gait irregularity is determined then a laser is activated. If no gait irregularity is determined, the laser is de-activated and the processor continues to receive motion data from the motion sensor.

A gait irregularity is determined by the algorithm performing a fast Fourier transformation on the motion data and by dividing it into predetermined frequency bands. A low frequency band relates to walking and a high frequency band relates to a gait irregularity. A mid-frequency band separates the high and low frequency bands to avoid false readings. The automatically activation of the cue is beneficial to prevent the user being concerned with having to activate the cue at a time when they are likely anxious, and ensures that a cue is provided for gait irregularities which the user may not have realised a gait irregularity was occurring or thought that the start of a gait irregularity wasn't of importance until too late. In the algorithm, a predetermined sensitivity number is used as a threshold to improve an accuracy in a determination of a gait irregularity. The predetermined sensitivity number is selectable and adjustable to provide a suitable sensitivity for a user wearing the apparatus.

A problem to this known apparatus is that a cue is produced in too many situations. Unnecessary cueing adversely affects an effectiveness of the cueing device in overcoming gait irregularities. Too much cueing may lead to habituation. To prevent habituation, cue production should be minimised to situations susceptible to gait irregularities in which a cue is really necessary and desired by the user.

In an article from Mazilu Sinziana et al. which is titled ‘the role of the wrist-mounted inertial sensors in detecting gait freeze episodes in Parkinson's disease’, pervasive and mobile computing, part 33, Jan. 13, 2016, pages 1-16, XP029841897, ISSN: 1574-1192, a correlation between wrist movements and a freezing of gait in Parkinson's disease, targeting FoG-detection from wrist-worn sensing data is investigated. Instead of placing a sensor on a lower limb, a focus is here on the wrist as an alternative placement. A comparison is made with state-of-the-art lower limb information used to detect FoG, and it is seen that the wrist increases the number of false detected events, while preserving the FoG hit-rate and detection latency. In a new smartphone based prototype an inertial measuring unit (IMU) is attached to an ankle and another inertial measuring unit to a wrist. A first model is used to process data from only the ankle, a second model from wrist motion and a third model uses both ankle and wrist data. If at least two of three consecutive classification outputs belong to the FoG category, the system considers a FoG is detected, and a rhythmic auditory stimulation starts.

Regarding the above-mentioned prior art, it is remarked that any discussion of documents, acts, materials, devices, articles or the like included in the present specification is for the purpose of providing a context for the present invention, and is not to be taken as an admission that any such matters form part of the prior art or were before the priority date of each claim or clause of this application common general knowledge in the field relevant to the present invention.

### SUMMARY OF THE DISCLOSURE

The general object of the present invention is to at least partially eliminate the above mentioned drawback and/or to provide a usable alternative. More specific, it is an object of the invention to improve an effectiveness of a cueing device by better distinguishing for situations requiring a cue, such that a cue is produced in situations which are susceptible to a gait irregularity and superfluous cueing is omitted in a situation not requiring a cue.

In the first aspect, according to the invention, this object is achieved by a cueing device. The cueing device is arranged for producing a cue for assisting a user during a motion. The motion can be a motion when a gait irregularity is determined. The cueing device comprises a control unit. The control unit is programmed by a first algorithm providing a first outcome to determine a gait irregularity, in particular a freeze of gait. Here, a gait irregularity determination means a recognition of an occurring gait irregularity or a prediction of an upcoming gait irregularity. The control unit is operatively connected to at least one motion sensor for receiving a sensor signal. Based on the received sensor signal, motion data is obtained by the control unit. By running the first algorithm on said motion data, a gait irregularity can be determined and is indicated by a positive outcome. If a gait irregularity is determined, the control unit may communicate an output signal to activate a cueing unit. The cueing unit is operatively connected to the control unit to provide a cue to the user to assist during the user's movement.

The cueing device according to the invention is improved in that the control unit is programmed by at least one additional algorithm providing an additional outcome based on motion data from said motion sensor to determine a gait irregularity in addition to the first outcome of the first algorithm. At least two outcomes are rendered by the control unit to indicate for a gait irregularity. The additional algorithm is different from the first algorithm. The additional algorithm has a different configuration. The additional algorithm deals with the motion data originating from the same motion sensor in a different manner and has different program rules. The additional algorithm may be based on a different formula for processing the motion data and/or include different parameters. The additional algorithm may include additional parameters to determine a gait irregularity. A determination of a gait irregularity may be an establishment or a prediction of an upcoming freeze of gait.

Preferably, the additional algorithm is running simultaneously with the first algorithm. At least one additional algorithm may be running in parallel with the first algorithm.

A cue is only provided in case an outcome of both the first algorithm and a second algorithm positively indicate by rendering positive outcomes in determining a gait irregularity. It is required that also the additional algorithm provides a positive indication of a gait irregularity. By running at least two gait irregularity algorithms, unnecessary cue production can be reduced which may prevent or defer habituation and increase the effectiveness of the cueing device. The additional algorithm is carried out to control the outcome of the first algorithm. By the additional algorithm, a gait irregularity is determined more accurately and reliable to generate a cue when it is desired. The more accurate determination of a gait irregularity is beneficial in allowing a user to receive a cue in more detailed distinguished situations. The motion data is analysed by both the first and second algorithm, which results in a plurality of markers indicative of different types of motion. Hence, the different types of motion are distinguished more precisely, such that a determination to provide a cue to the user can be based on more detailed supporting data.

In comparison with the prior art investigation which is disclosed in the article of Mazilu et al. according to the invention, obtained motion data from the same motion sensor is processed before establishing or predicting a gait irregularity e.g. a FoG. In Mazilu et al. different motion data originating from two different motion sensors, i.e. an ankle sensor or a wrist sensor, is processed by three FoG detection models. According to the invention, motion data which is obtained by a single motion sensor, in particular a knee motion sensor, is processed by more than one algorithms. Preferably, the same motion data is processed at least two times by at least two different algorithms to determine a gait irregularity.

In an embodiment of the cueing device according to the invention, the control unit is programmed with at least three different algorithms for each determining a gait irregularity. Each algorithm provides an own outcome in determining a gait irregularity. The control unit may be programmed to include a first, second and third algorithm. The control unit may be programmed to provide an output signal to the cueing unit to provide a cue to the user only in case that all algorithms positively indicate for a gait irregularity. A required amount of algorithms which have to positively indicate for a gait irregularity before providing a cue may be adjustable.

In an embodiment of the cueing device according to the invention, the control unit is programmed with at least three different algorithms for each determining a gait irregularity. At least one algorithm is selectable to be taken into account or cancelled, such that a variety of combinations of algorithms are available. A select value can be programmed in dependence of an occurring circumstance or to be disabled by a user or a physician to indicate which algorithms are to be taken into account in determining a gait irregularity. Herewith, the cueing device can be adapted to a personal situation. A personalised cueing device is provided. In a particular circumstance—e.g. a noisy or dark situation—it may be indicated that the first and third algorithm should run to provide a cue, while in another circumstance—e.g. a silent or visual bright situation—, it may be indicated that the first and second algorithm should run to provide a cue. A particular algorithm may run for a selected time period, e.g. an afternoon, while another algorithm may run for another time period, e.g. an evening. Herewith, particular combinations of algorithms are available to be carried out in particular circumstances. Beneficially, dedicated combinations of algorithms may contribute in more accurate determined gait irregularities typically occurring in particular circumstances.

In an embodiment of the cueing device according to the invention, at least one of the algorithms is programmed to include at least one environmental parameter, e.g. shoe type, walking aid, hard or soft floor-surface, indoor, outdoor, a location, temperature and/or humidity. The environmental parameter may typically change on a daily basis. The cueing device may include a sensor for measuring the environmental parameter. All algorithms may include an environmental parameter, but preferably, at least one of the algorithms is distinguished from another algorithm by including an environmental parameter while the other algorithms lacks such environmental parameter.

In an embodiment of the cueing device according to the invention, the control unit is programmed with at least three different algorithms for each determining a gait irregularity. At least one algorithm is selectable to be taken into account or cancelled, such that a variety of combinations of algorithms are available. A select value can be programmed in dependence of personal parameters, e.g. a persons length, BMI, stride length, gait cycle, or arranged to be disabled by a user or a physician to indicate which algorithms are to be taken into account in determining a gait irregularity for a particular person. Such a selection of personal parameter may typically be constant over an amount of time, in particular for at least some weeks. For a specific person—e.g. a tall person—it may be indicated that the first and third algorithm should run to provide a cue, while for another person—e.g. a small person—, it may be indicated that the first and second algorithm should run to provide a cue. Herewith, the cueing device provides particular combinations of algorithms which are available to be carried out to better distinguish for different users. Beneficially, dedicated combinations of algorithms may contribute in more accurate determined gait irregularities typically relating to personal parameters.

In an embodiment of the cueing device according to the invention, at least one of the algorithms is programmed to include at least one user dependent parameter. At least one of the algorithms includes a user profile. The user profile contains at least one user dependent parameter. A user dependent parameter may include at least one of the parameters: a body length, a body weight, a BMI, and a stride length. Preferably, at least one of the user dependent parameters is adjustable by the user.

In an embodiment of the cueing device according to the invention, the cueing device has a user interface which is operatively connected to the control unit to allow an adjustment of an amount of algorithms being necessary to positively indicate for a gait irregularity to provide a cue to the user. The user interface may be accessible by the user or by a physician. Preferably, the user interface is accessible by the physician only. The user interface may be formed by a switch button or include a display to allow the user to switch between operative modes corresponding with a certain amount of algorithms being involved in determining a gait irregularity. The user interface of the cueing device may be formed by a remote connected device, like a smart phone or smart watch, to allow a remote adjustment of a set minimum amount of algorithms positively indicating a gait irregularity required to provide a cue.

In an embodiment of the cueing device according to the invention, the cueing device includes a user interface to allow a person to select a predefined personal user setting based on an intended activity. The intended activity may for example be an indoor activity or an outdoor activity. The cueing device including this functionality allows a user to select an operation of the cueing device based on predetermined settings for an intended use.

In an embodiment of the cueing device according to the invention, the control unit is further programmed by a routine algorithm to determine a type of motion of the user based on a received sensor signal. The routine algorithm is distinguished from other algorithms by a classification step in which motion data is classified as relating to a routine or a non-routine motion. Routine motion considers a motion which is carried out by the user without or with less attention of the user, e.g. distance walking. In a routine motion, a user has no particular focus in carrying out the motion. A non-routine motion considers a motion of the user which is more incidental and which requires more attention of the user, e.g. stair climbing. The routine algorithm is preferably carried out before running any of the available algorithms to determine a gait irregularity. Preferably, the control unit is programmed that if a non-routine motion is determined, a running of any of the available gait irregularity algorithms is cancelled. A cancellation of the gait irregularity algorithms may be beneficial in saving battery of the device by not processing motion data in a situation which is less vulnerable to a gait irregularity.

The application of a routine algorithm is independent of an amount of gait irregularity determining algorithms being programmed in a cueing device. According to a side-aspect of the invention, the routine algorithm to distinguish a routine or non-routine motion before a possible start of an algorithm to determine a gait irregularity is programmed in a control unit in an embodiment of a cueing device which includes only a single algorithm to determine a gait irregularity. The routine algorithm may be carried out before running the single algorithm or multiple algorithms to determine a gait irregularity.

In an embodiment of the cueing device according to the invention, the cueing device is a wearable cueing device which is formed by a system comprising a combination of a smartwatch and a smartphone, wherein the smartphone functions as a housing provided with at least one motion sensor for providing a sensor signal, and wherein the smartwatch functions as a cueing unit for producing a cue to the user in case of a gait irregularity. The smart phone and the smartwatch are each programmed with an app which is configured to establish a cooperation of the combination of the smart phone and smartwatch to determine and to produce a cue in case of a gait irregularity. In use, the smartwatch is worn on a wrist and the smartphone is positioned at one of the legs of the user. The smart phone may be simply inserted in a hose pocket, or may be attached to the leg of the user by a limb band. By forming the wearable cueing device by commonly available devices, a wearable cueing device is easily available for a person with Parkinson. Beneficially, only a smartwatch app and a smartphone app needs to be downloaded and installed by the user to obtain the wearable cueing device.

In an embodiment of the cueing device according to the invention, the cueing device is a wearable cueing device comprising a strap and a housing for housing the motion sensor, wherein the housing is connectable to the strap.

In an embodiment of the cueing device according to the invention, the cueing device further comprises an alarming unit to generate an alarm to attention the user and/or a remote alarm to attention a user-related person, like a relative or a physician. The cueing device may be a wearable fall alarming device including a cueing unit and an alarming unit which is configured to produce an alarm in case of a determination of a risk of falling instead of producing a cue as described above regarding the cueing device. The wearable fall alarming device may comprise multiple algorithms to provide multiple outcomes in determining a risk of falling by determining a gait irregularity. The control unit is programmed by at least a first and second algorithm providing at least a first and second outcome to determine a gait irregularity, wherein an alarm is only produced in case that the at least first and second outcomes of the at least first and second algorithms positively indicate for a gait irregularity.

Further, the invention relates to a computer-implemented control method for controlling a cueing device. The cueing device is configured to provide a cue during a motion of the user. According to the invention, the cueing device control method comprises a step of receiving a sensor signal from at least one motion sensor. A step of acquiring motion data based on the received sensor signal. A step of running a first algorithm on the motion data to determine a gait irregularity. A step of running at least a second algorithm on the same motion data to determine a gait irregularity. And a step of providing a cue only if at least two algorithms each have an outcome positively indicating for a gait irregularity.

Further, the invention relates to a computer program product comprising a computer readable medium. The computer readable medium as computer readable code embodied therein. The computer readable code is configured such that, on execution by a suitable computer or processor, the computer processor is caused to perform the method according to the invention.

Further, the invention relates to a method, in particular a computer-implemented method for controlling a cueing device formed by a combination of a smartwatch and a smartphone which combination is configured to provide a cue during a motion of a user, the method comprising:
receiving a sensor signal from at least one motion sensor of the smartphone;
acquiring motion data based on the received sensor signal by a control unit being formed by a processor of one of the smartwatch and smartphone;
running a gait irregularity algorithm on the motion data to determine a gait irregularity;
in particular, running at least a second gait irregularity algorithm on the same motion data to determine a gait irregularity;
in particular, providing a cue only if at least two algorithms have an outcome positively indicating for a gait irregularity.

In an embodiment of the cueing device according to the invention comprising a smartphone and a smartwatch, the method further comprises the step of producing a cue by one of the smartwatch or smart phone. Preferably, a cue is produced by the smartwatch, wherein the smartphone is used to obtain motion data.

Thus, a cueing device is provided for producing a cue in assisting a user during a motion. The cueing device comprises a control unit operatively connected with a motion sensor and a cueing unit. The control unit is programmed with multiple algorithms providing multiple outcomes in determining a gait irregularity during the motion. A cue is only produced in case outcomes of at least two algorithms positively indicate for a gait irregularity. Cue production should be minimised to situations in which a cue is really necessary and desired by the user. Different situations and personal specifics can be better distinguished by using the at least two outcomes of the at least two gait irregularity algorithms. Unnecessary cue production can be reduced which prevents habituation and increases the effectiveness of the cueing device.

In a first side-aspect of the first aspect according to the invention, instead of a cueing device, a wearable fall alarming device is provided which is configured to produce an alarm in case of a determination of a risk of falling instead of producing a cue as described above regarding the cueing device. The wearable fall alarming device has an alarming unit to generate an alarm to attention the user and/or a remote alarm to attention user related persons, like a relative or a physician. The wearable fall alarming device according to the first side-aspect of the invention comprises multiple algorithms to provide multiple outcomes in determining a risk of falling. Embodiments of the wearable fall alarming device are described in the appended claims in which a cueing unit is to be considered as substituted by an alarm unit.

In a second aspect, the invention relates to a cueing device comprising a cueing unit for producing a user perceptible cue, a sensor for obtaining motion data based on sensor information, a control unit operatively connected with the cueing unit and the sensor in which the control unit is programmed to start the cueing unit based on obtained motion data. The cueing device is configured to include a self-activation by the user to allow a user to start the cueing unit at a moment that the user is aware of an occurring or upcoming gait irregularity.

US2009099627A1 is directed towards controlling therapy delivery to a patient based on a determination of whether a patient is in a movement state based on a brain signal of the patient. A therapy parameter value is changed upon determining the patient is in the movement state.

A therapy system including a sensing device and an external cue device is disclosed. Rather than requiring a patient to manually activate the external cue device, the therapy system automatically activates external cue device in response to a sensed state, condition or event.

After the sensing device determines that the patient is in a movement state, external cue device may deliver a sensory cue, such as a visual, somatosensory or auditory cue, to the patient in order to help control the movement disorder. Automatic activation of the external cue device may help to provide the patient with a better control and timing of therapy delivery by the external cue device by eliminating the need for the patient, who exhibits some difficulty with movement, to manually activate external cue device. In addition, automatically initiating the delivery of a sensory cue in response to detecting a movement state may enable the therapy system to minimize the time between when the patient needs the therapy and when the therapy is actually delivered.

The therapy system provides a responsive system for controlling the delivery of therapy to the patient. As one example of the responsiveness of therapy system, the therapy system times the delivery of therapy to the patient such that the patient receives the therapy at a relevant time, i.e., when it is particularly useful to patient. In contrast, an external cue device that requires patient to purposefully initiate the delivery of a sensory cue by interacting with an input mechanism may be less useful. For example, if the patient exhibits motion impairment, the patient may find it difficult to initiate the movement to activate the external cue device (e.g., via a button or another input mechanism).

A disadvantage of the automatic activation by this therapy system is that it requires a brain signal of the patient. The brain signal may include a bioelectrical signal, such as an EEG signal, an ECoG signal, a signal generated from measured field potentials within one or more regions of a patient's brain and/or action potentials from single cells within the patient's brain. Obtaining such a brain signal to predict a movement state makes the therapy system rather complex and less suitable for a daily life usage.

EP3.283.039 is published in 2018 and refers to US2009/0099627. In an introduction, it is mentioned that cueing methodologies can improve gait performance in Parkinson disease populations. These cues facilitate an initiation and continuation of repetitive sequential movements such as gait and can assist in handling, or even overcoming, gait irregularities, in particular freezing of gait episodes. Devices are known which aim to stop gait irregularities, while other devices provide cues aiming to prevent gait irregularities. The latter devices are activated (manually or automatically) when motion is detected, and provide continuous cueing, by for example repetitive sound from a metronome being delivered via an earpiece.

In the disclosure, a gait management apparatus is described which comprises at least one motion sensor for capturing gait information, at least one cueing actuator and a controller to activate the cueing actuator automatically upon a detection or a potential gait dysfunction. The controller is configured to determine characteristics of the patient and to activate cueing customised to the patient.

A simple embodiment of the apparatus is disclosed in which an interface including a button which is provided for a manual activation. Instead of this button, another embodiment is disclosed in which the controller is configured to recognise at least one tap on an enclosure of the controller as a patient trigger to activate cueing. The controller may be configured to be customised to parameters specific to a patient for recognising the taps. For example, the control may be configured to recognise a series of two taps as a patient trigger.

US2020/009545 discloses a structure of a cueing device to be mounted to a mobile walker which also includes a manual activation. The cueing device comprises an activation switch for activating an audible cueing. An activation module is arranged inside a box-shaped housing including the activation switch at an outside of the housing.

As already mentioned in US 2009/0099627, a problem to this kind of manual activation by tapping or pressing a button is that when a patient exhibits a motion impairment, the patient may find it difficult to manually activate a cue device via the button or another input mechanism. Regarding the above-mentioned prior art, it is remarked that any discussion of documents, acts, materials, devices, articles or the like included in the present specification is for the purpose of providing a context for the present invention, and is not to be taken as an admission that any such matters form part of the prior art or were before the priority date of each claim or clause of this application common general knowledge in the field relevant to the present invention.

The general object of the present invention is to at least partially eliminate the above mentioned drawback and/or to provide a usable alternative. More specific, it is an object of the invention to provide a cueing device including an effective and user-friendly self-activating possibility to start cueing in any situation.

In a second aspect, according to the invention, this object is achieved by a cueing device according to clause 1 hereafter.

According to the invention, a cueing device for assisting a user during a motion by producing a user perceptible cue is provided which comprises a cueing module, at least one motion sensor and a control unit. The cueing unit is arranged for producing a cue to the user. In particular, the cueing unit is arranged to produce an audible and/or a haptic cue. The audible cue is configured to encourage a walking process of the user, e.g. by providing a rhythmic sound. The at least one motion sensor is arranged to provide a sensor signal to the control unit to obtain motion data. The at least one motion sensor is adapted to generate sensory information responsive to a movement of the user. The control unit is adapted to process the sensory information. The control unit is operatively connected with the cueing unit and the at least one motion sensor to provide the cue based on a determined gait irregularity analysed from the motion data. This is also called automatic cueing when determining a gait irregularity. Based on the obtained motion data, in a determination by the control unit, an occurring gait irregularity can be established or an upcoming gait irregularity can be predicted. The control unit is further programmed with a self-activation function. The self-activation function means that the user can start a generation of a cue by the cueing unit when desired.

According to the second aspect of the invention, an improvement is obtained in that the self-activation function of the cueing device is based on a detection of a hands-free predetermined user input for activating the cueing unit. The self-activation function may also be called a handsfree self-initiated cueing or a handsfree user-start cueing. The predetermined user input is an input given by the user which might be a person wearing the cueing device or another involved person, like a partner or practitioner. The predetermined user input to be recognised by the cueing device serves as a starting signal to start cueing. The predetermined user input is detected by at least one sensor of the cueing device. The at least one sensor for detecting the predetermined user input is operatively connected to the control unit. Preferably, the at least one motion sensor which is used to obtain motion data of the user is also used to detect the predetermined user input. In that case, the user input is a predetermined user motion.

The self-activation by a predetermined hands-free user input is beneficial, because the cueing unit can be started handsfree by a person wearing the cueing device or by an accompanying person, like a partner, by carrying out the handsfree user-input without a need for manually operating the cueing device. A non-manual predetermined user input is beneficial, because a manual input would require a precise finger movement which is difficult to carry out in case of a gait irregularity. The non-manual predetermined user input, e.g. making a call ‘start cue’ or a foot stamp, can be easily carried out by the user even in case of an occurring gait irregularity. Herewith, the user can activate a cue in all kind of situations. Preferably, the predetermined user input is recordable on the cueing device by the user himself. The user can teach the cueing device a self-chosen user input.

The predetermined user input may be defined during a therapeutic session to find out which user input is working optimal for a particular user. The cueing device may include a learning program to be used during the therapeutic session in which the learning program provides a functionality to record a user input. The recorded user input is beneficially more specific for a certain user than a preprogrammed user input. Advantageously, the use of a recorded predetermined user input instead of a preprogrammed predetermined user input may result in a more accurate determination of a gait irregularity.

Preferably, the predetermined user input is a selection of at least one of a body waddle motion, a walking on the place, or a heel tap motion. During empirical experiments, these predetermined user inputs did provide optimal results in determining a gait irregularity.

In an embodiment of the cueing device according to the invention, the cueing device includes both a manual and a handsfree self-activation. Acquiring the user input by a sensor operatively connected to the control unit to start operating the cueing unit may make a manual mechanical input operation by pressing a button or tapping a touch panel as disclosed in the prior art redundant, but can be embodied in addition to manual activation. The handsfree self-activation is in particular beneficial in cases of trembling or an occurring freeze of gait. A further benefit of the hands-free self-activation is that another person, like a partner of the user, may activate the cueing device, e.g. by saying a start-command, when that person observes an event in which a cueing is desired. Thus, the control unit may be programmed to recognise a predetermined user input which input originates from another person than the user self.

In an embodiment of the cueing device according to the invention, the predetermined user input may be a user sound. The user sound is a sound produced by the user which is considered to be the patient wearing the cueing device or an accompanying person, like a partner. The user sound may be a spoken command like ‘cue start’, a whistling or tongue clacking sound. Just calling for a cue by mentioning the predetermined word or making a clacking sound suffices to start the cueing unit to produce a user perceptible cue to help the user to prevent or minimise a gait irregularity. The cueing device includes a microphone which is operatively connected to the control unit. The user sound is detected by the microphone of the cueing device.

Producing the predetermined user sound without a need to accurately position a hand, e.g. to press a button, is beneficial, because the user may now hold his hands where they are at a moment that the user feels a gait irregularity upcoming. When the user is aware of an upcoming gait irregularity, the user may stay in a stable position and may keep for example his hands on the grips of a walker device at the moment of an instability caused by the gait irregularity.

In an embodiment of the cueing device according to the invention, the predetermined user input is a predetermined user motion. The predetermined user motion is a motion which is carried out by the user and pre-programmed in the control unit to be recognised to start the cueing unit. The predetermined user motion is recorded in the memory of the cueing device in which the memory is operatively connected to the control unit. The predetermined user motion is detectable by the at least one motion sensor of the cueing device.

A predetermined user motion to activate the cueing device is beneficial in comparison with a sound, because a Parkinson patient may have a difficulty in speaking. Besides that a motion may be less vulnerable to an environment in which a specific sound may get disturbed from a surrounding noise. Carrying out a predetermined user motion might be less disturbing for a social environment of the user. A user can carry out the predetermined user motion, e.g. a foot stamp, without interrupting a conversation with another person.

Preferably, the predetermined user motion is a leg motion. A predetermined user leg motion may for example be formed by elevating a leg, stretching a leg, heel or toe tapping, bending a knee or a weighting over to a left or right leg. Preferably, the predetermined user leg motion is a heel tap which has proven by experiments a surprisingly effective activation motion. In addition, the heel tap appears most doable for most patients. The cueing device may be a device mounted to a walking aid, like a stick or wheeled walker. Preferably, the cueing device is a wearable cueing device which is attachable to a human's body, because such a wearable may more precisely detect the user leg motion. For example, the wearable cueing device may include a wrist unit, e.g. a smartwatch, to be attached to a user's wrist. Preferably, to obtain an accurate user leg motion, the wearable cueing device comprises a strap for attaching the cueing device to a user's lower extremity, e.g. just below a user's knee at a peroneus compartment.

Preferably, the at least one motion sensor is arranged for an attachment to a user's leg. In particular, the at least one motion sensor is connected to a strap for attaching the at least one motion sensor to a leg of the user. This leg attached motion sensor can be used to obtain motion data and can also be used to detect the predetermined user motion. The positioning of the motion sensor at a user's leg and a user leg motion as a predetermined user motion for starting the cueing unit is beneficial to obtain an accurate operation which is hardly susceptible to misconceptions by the control unit.

In an embodiment of the cueing device according to the invention, the cueing device comprises a memory for storing motion data measured by the at least one motion sensor. The stored motion data is user specific motion data. The stored motion data may include a time stamp. The stored motion data may be analysed to determine a suitable predetermined user motion, which is to be programmed for self-activation of the cueing unit. The stored motion data can be analysed to consider which typical movement during a short time interval, e.g. 1 or 2 seconds, precedes before a happening of a gait irregularity. This typical movement is useful to determine a user motion for self-activation. In a step, it may be analysed which body parts are involved during this typical movement to understand which remaining muscles are suitable to produce the predetermined user motion. Herewith, a suitable user motion is determined to be programmed in the cueing device as being the predetermined user motion for self-activation of the cueing unit. Beneficially, the predetermined user motion can be selected to be a user motion which can be easily carried out by the user when the user is aware of an upcoming gait irregularity.

In an embodiment of the cueing unit according to the invention, the predetermined user motion is a therapeutical user motion. The therapeutical user motion is a user motion which is implemented in the cueing device based on a physician's instruction. The therapeutical user motion is a user motion instructed to the user by a physician. In particular, the therapeutical user motion is a selectable user motion which fits to a user situation. The physician can select a particular user motion to be implemented in the cueing device which fits best on how a gait irregularity manifests. The selection may be made out of a group of pre-programmed user motions. A therapeutical user motion may for example be selected form a group containing a toe tap, a heel tap, a foot stamp, a knee bend, a leg raise and/or a leg stretch. Preferably, the therapeutical user motion is a heel tap which has proven by experiments a surprisingly effective activation motion. In addition, the heel tap appears most doable for most patients.

In an embodiment of the cueing device according to the invention, the control unit is programmed by an algorithm to recognise the therapeutical user motion to start the cueing unit. The control unit may be programmed by at least one additional algorithm having a separate outcome for detecting a particular user motion to start the cueing unit. Preferably, the control unit is programmed in that when at least any of the algorithms for recognising the therapeutical user motion has a positive outcome in any case starts the cueing unit to produce a cue. Herewith, multiple algorithms having each a separate outcome can run in parallel or in series to obtain an accurate detection to cover a variety of situations in which a gait irregularity may occur.

In an embodiment of the cueing device according to the invention, the cueing unit is arranged to produce an audible cue. In comparison with a tactical or visual cue, an audible cue is advantageous, because an audible cue can be varied more easily and tuned to a particular occurring situation.

Preferably, the cueing unit is configured to initiate an audible cue by a randomly selected sound. The randomly selected sound provides an unexpected cue to the user. In combination with a self-activation function of the wearable cueing unit, the randomly selected initial sound is beneficial to keep a surprising effect of the cue. Such a surprising effect increases an effectiveness of the activation. Although the user activates the cue by himself, which might reduce a surprising effect of the queue, the unexpected tune of the initial sound might compensate this which contributes to a proper working of the cue.

In an embodiment of the cueing device according to the invention, the cueing unit is arranged to produce an initial tactile cue by a randomly selected vibration.

In an embodiment of the cueing device according to the invention, the control unit includes a self-deactivation of the cueing unit based on a detection of a regular gait. The self-deactivation of the cueing unit may be an automatic de-activation, e.g. based on a time interval after an end of a gait irregularity. In particular, the control unit is further programmed to include a self-deactivation of the cueing unit by a predetermined user input which is preferably a predetermined user motion, more preferably a hands-free predetermined user motion. Preferably, the automatic de-activation is based on a therapeutic user motion.

Further, the invention relates to a computer-implemented control method for controlling a cueing device for assisting a user during a motion by producing a cue, wherein the method comprises the steps of:
receiving a sensor signal from at least one motion sensor;
acquiring motion data based on the received sensor signal;
recognising a user input from obtained sensory data;
comparing the user input with a predetermined user input;
producing a user perceptible cue by activating a cueing unit.

Preferably, the at least one motion sensor is used to obtain the sensory data from which the user input is recognised to start the cueing unit.

Thus, a method and a cueing device is provided for assisting a user during a motion when determining a gait irregularity. The cueing device has a cueing unit for producing a cue, at least one motion sensor for providing a sensor signal to obtain motion data and a control unit operatively connected with the cueing unit and the at least one motion sensor to provide a cue based on a determined gait irregularity. The cueing device has a self-activation function to allow the user to initiate a cue on an own initiative. The self-activation function is based on a hands-free predetermined user input which is detectable by a sensor of the cueing device. The hands-free predetermined user input may be a sound or a specific user motion. Preferably, the user motion is a lower limb motion, for example a toe tap, which can be carried out easily despite of an occurring gait irregularity.

Embodiments according to the invention are defined by the following clauses:

1. Cueing device (1) for assisting a user during a motion by producing a user perceptible cue when determining a gait irregularity, wherein the cueing device comprises:
a cueing unit for producing a cue to the user;
at least one motion sensor for providing a sensor signal to obtain motion data;
a control unit operatively connected to the cueing unit and the at least one motion sensor to provide a cue based on a determined gait irregularity after analysing the motion data, wherein the control unit is programmed with a self-activation function based on a detection by a sensor of a hands-free predetermined user input for activating the cueing unit.

2. Cueing device according to clause 1, wherein the predetermined user input is a predetermined user sound and wherein the sensor for detecting the predetermined user sound is a microphone.

3. Cueing device according to clause 1 or 2, wherein the predetermined user input is a predetermined user motion which is carried out by the user to activate the cueing unit, wherein the predetermined user motion is detectable by the at least one motion sensor of the cueing device.

4. Cueing device according to clause 3, wherein the predetermined user motion is a leg motion.

5. Cueing device according to clause 4, wherein the leg motion is at least one of a motion selected from the following:
raising a leg;
stretching a leg;
tapping by a toe or heel;
a foot stamp;
weighting over to one leg; and
bending one or both knees.

6. Cueing device according to any of the clauses 3-5, wherein the predetermined user motion is a therapeutical user motion which is a user motion instructed to the user by a physician, in particular a selectable user motion fitting to user's situation, wherein the predetermined user motion is preferably a recorded predetermined user motion instead of a preprogrammed predetermined user motion.

7. Cueing device according to any of the preceding clauses, wherein the cueing device comprises besides the self-activation function based on a detection by a sensor of a hands-free predetermined user input for activating the cueing unit also a mechanical input means, like a press button or a touch panel, for a manual self-activation.

8. Cueing device according to any of the preceding clauses, wherein the control unit includes a self-deactivation of the cueing unit based on a detection of a regular gait.

9. Cueing device according to any of the preceding clauses, wherein the control unit is further programmed to include a self-deactivation of the cueing unit by a predetermined user input which is preferably a predetermined user motion, more preferably a hands-free predetermined user motion.

10. Cueing device according to any of the preceding clauses, wherein the control unit is programmed to include an automatic deactivation of the cueing unit, in particular a deactivation based on a time interval after an end of a gait irregularity.

11. Cueing device according to any of the preceding clauses, wherein the cueing unit initiates an audible and/or tactile cue by a randomly selected sound and/or vibration.

12. A computer-implemented method for controlling a cueing device configured to provide a cue during a motion of the user when determining a gait irregularity, the method comprising:
receiving a sensor signal from at least one motion sensor;
acquiring motion data based on the received sensor signal;
recognising a user input from obtained sensory data;
comparing the user input with a predetermined user input;
producing a user perceptible cue by activating a cueing unit in case the user input corresponds with the predetermined user input hoe.

13. Method according to clause 12, wherein the at least one motion sensor is used to obtain sensory data from which the user motion is recognised.

14. A computer program product comprising a computer readable medium, the computer readable medium having computer readable code embodied therein, the computer readable code being configured such that on execution by a suitable computer or processor, the computer or processor is caused to perform the method according to clause 12 or 13.

Summarised, in an embodiment according to the second aspect, a cueing device for assisting a user during a motion when determining a gait irregularity is provided. The cueing device has a cueing unit for producing a cue, at least one motion sensor for providing a sensor signal to obtain motion data and a control unit operatively connected with the cueing unit and the at least one motion sensor to provide a cue based on a determined gait irregularity. The cueing device has a self-activation function to allow the user to initiate a cue on an own initiative. The self-activation function is based on a hands-free predetermined user input which is detectable by a sensor of the cueing device. The hands-free predetermined user input may be a sound or a specific user motion. Preferably, the user motion is a lower limb motion, for example a toe tap, preferably a heel tap, which can be carried out easily despite of an occurring gait irregularity.

According to a third aspect of the invention, the invention relates to a wearable cueing device for assisting a user during a motion by producing a cue. The wearable cueing device comprises a strap for releasably carrying a housing for electronics.

People suffering Parkinson's disease, simply called Parkinson, have symptoms of shaking, rigidity, slowness of movement and difficulty with walking. During a motion, a gait irregularity may occur. Festinating gait is a typical gait including an involuntary acceleration manifested by shortening shuffling steps followed by a forward tilt of a person's body. Freezing of Gait is another symptom of Parkinson which is characterised by an absence or marked reduction of forward progression of the feet despite an intention to walk.

Cueing is a scientifically proven technique that has proven to improve a gait of a person with Parkinson. A visual, auditory or haptic cue may be produced by a wearable cueing device to help the person in preventing gait irregularities.

KR20210069918 relates to a gait compensator for patients with degenerative brain disease, which can protect the patient by inducing stable gait by assisting gait by alleviating leg tremor that causes gait freezing in patients with degenerative brain disease. To this end, a gait compensator for a patient with degenerative brain disease is installed on a band or a protector worn under the patient's knee to correct the patient's leg tremor, a sensor unit installed on the body to detect the patient's leg tremor. A tremor detecting unit that measures the magnitude and direction of a leg tremor according to a signal, a tremor correcting unit that determines the magnitude and direction of a leg tremor opposite to the magnitude and direction data of the leg tremor detected by the tremor detecting unit, and the tremor correcting unit and a control unit for receiving the determined data from and operating the motor unit to apply vibration and force in the opposite direction to the leg tremor.

WO2021004680 discloses a portable device for mobilizing a person suffering from a neurological gait dysfunction as a result of impaired proprioception, and carrying aid for such a device. The carrying aid is arranged to carry a gait assist unit at both a left and right leg. Each gait assist unit includes a motion sensor. The carrying aid includes a horizontally extending strap which is configured to position the motion sensor above a knee level at an outside leg position.

U.S. Ser. No. 10/242,590 discloses a wearable gait training device for gait rehabilitation. The training device includes a power supply unit, a light emitting unit, a support strap and a trigger unit. The power supply unit and the light emitting unit are coupled with each other and disposed on the support strap attached on a position below the knee joint. The support strap is provided with buckling structures forming a strap closure disposed at two ends of the strap which may include a Velcro tape.

Such a wearable cueing device is frequently put on and off by the user. For that, it is desired to provide a user-friendly wearable which can be quickly placed in position and attached in a manner which warrants a proper functioning of the cueing device.

Regarding the above-mentioned prior art, it is remarked that any discussion of documents, acts, materials, devices, articles or the like included in the present specification is for the purpose of providing a context for the present invention, and is not to be taken as an admission that any such matters form part of the prior art or were before the priority date of each claim or clause of this application common general knowledge in the field relevant to the present invention.

The general object of the present invention is to at least partially eliminate the above mentioned drawback and/or to provide a usable alternative. More specific, it is an object of the invention to provide a wearable cueing device which is user-friendly in correctly putting on the cueing device to obtain accurate measurements.

In the third aspect according to the invention, this object is achieved by a wearable cueing device according to following clause 1.

According to the third aspect, a wearable cueing device including a strap, a housing and a cueing unit is provided. The wearable cueing device is arranged to assist the user during a motion by producing a cue. The wearable cueing device comprises a cueing unit for producing a cue to the user in case of a gait irregularity, in particular a freeze of gait. Preferably, the produced cue is an auditory cue.

The strap of the cueing device is arranged to carry the housing. The strap is configured to encircle a user's leg and has a closure, for example a buckle or a Velcro strap end, for closing and releasing the strap. In particular, the strap is a knee strap which is configured to enclose a leg just below a knee of the user, in particular onto a peroneus longus.

The housing is arranged for housing electronics. The electronics include at least one motion sensor, in particular a gait sensor, for detecting a motion of the user. The motion sensor provides a sensor signal to a control unit to obtain motion data to establish or predict a gait irregularity. In case of a determination of a gait irregularity, the control unit is programmed to provide an output signal to the cueing unit to produce a cue to the user.

The housing has a predetermined upwards orientation for a correct placement of the at least one motion sensor with respect to a user's leg. The upwards orientation of the housing may be marked by a marker, like an arrow or other symbol.

Preferably, the predetermined upwards orientation is defined by an intuitive form of the housing. The form of the housing intuitively tells a user how to orient the housing with respect to the users leg to attach the housing to a strap around the users leg. The form of the housing follows its function. Preferably, the housing has an arrow shape which is formed by a flat upper side and a converging bottom side, in particular a rounded bottom side. The converging bottom side forms an arrowhead. The converging bottom side tells the user which side should be inserted into an opening for attaching the housing to the strap. Herewith, the converging bottom side forms an insertion side of the housing for attaching the housing by an insertion into a pocket of the strap.

According to the invention, the strap is improved by a holder fixed to the strap which holder is arranged to carry the housing. The holder of the strap forms an insert pocket which has an insertion upper opening and an insertion lower opening for slidably receiving the housing in the holder. The insert upper opening and the insert lower opening are equally sized allowing an insertion of the housing through either the upper or lower opening.

The housing is manually attachable and releasable from the holder by a sliding movement. The manual release of the housing from the strap may serve for charging or cleaning the cueing device, for connecting the cueing device to a computer etc. In case of a Parkinson wearable cueing device, a particular placement of the cueing device to the one or other leg is often prescribed. A particular Parkinson patient may carry the cueing device at a left leg, while another Parkinson patient may carry the cueing device at a right leg. A change of leg should not disturb a proper working of the Parkinson cueing device.

Due to the insertion opening at both an upper side and a bottom side, the strap can be closed around a leg in an upside down manner without affecting the way a user or a caregiver is familiar to insert the housing into the holder. Most of the times, when putting on the cueing device, a user will intuitively position the closure of the strap at an outside of his leg. Hence, depending on which leg the user attaches the strap, whilst keeping the closure to the outside, the strap will get oriented up- or downwards. Due to the open upper and bottom side of the holder, it does not matter on which left or right leg the user has fasten the strap, the housing can always be shifted into the holder from above. The user or physician will always hold the housing in the predetermined upwards orientation and can then insert the housing in always a same shifting manner into the holder of the strap which is independent of the attachment of the strap to a left or right leg. Herewith, this feature contributes to a proper and reliable working of the wearable cueing device. As the housing has a predetermined upwards orientation being required for an accurate operation of the cueing device, the design of the holder of the strap allowing to receive the housing always in a same manner contributes to an increased safety in using the cueing device in that the housing will be attached to the strap in a correct manner.

In an embodiment of the wearable cueing device according to the invention, the holder of the strap comprises a carrying loop. The carrying loop is preferably made of an elastic material, in particular an elastic textile material. The carrying loop is configured to receive the housing. The carrying loop forms an insert pocket for receiving the housing. The insert pocket has a top and bottom pocket opening for inserting the housing into the insert pocket. Preferably, after placement of the housing, the bottom side of the housing has passed through the bottom pocket opening. The housing has a housing length which is larger than a carrying loop height. An upper portion and a lower portion of the housing is visible after placement of the housing behind the carrying loop which further contributes to the safety of the cueing device in that a user or physician may have a visual indication of a correct orientation of the housing.

In an embodiment of the wearable cueing device according to the invention, the housing is attachable to the holder by a form-fit. In contrast to a friction-based clamping, the form-fit provides a precise positioning of the housing with respect to the strap. Herewith, an accurate positioning of the housing, in particular of the at least one motion sensor, is increased. To determine a gait irregularity for a Parkinson patient with a high reliability, a precise positioning of the motion sensor is highly desired. For that reason, the precise positioning of the housing by the form-fit is in particular beneficial for a Parkinson cueing device.

In an embodiment of the wearable cueing device according to the invention, the rear side of the housing may include a form member and the holder may include a complimentary member which engage to each other after placement of the housing into the holder. Advantageously, the form member and complementary member increase a positional accuracy of the housing relative to the strap, in particular with respect to the strap closure. Herewith, the complementary member of the holder defines a lateral position of the housing with respect to a user's leg. Preferably, the complementary member may further lock an up- or downwards position of the housing with respect to the strap. Together with the elastic carrying loop, the form member and the complementary member may form a snap-fit of the housing into the insert pocket. A further advantage of the arrangement of the form member and the complementary member is that a large portion of the rear side of the housing may get in contact with a user's leg which may be beneficial in transferring a haptic cue to the users leg.

In an embodiment of the wearable cueing device according to the invention, the rear side of the housing is provided with a housing recess for receiving a strap elevation. The strap elevation is received in the housing recess when the housing is shifted into the holder of the strap. An engagement of the strap elevation and the housing recess further contributes to a precise positioning of the housing with respect to the strap which is beneficial in increasing an accuracy of a measurement by the motion sensor. In addition, the engagement may be further beneficial in transferring a haptic cue to the user.

The insert pocket has a pocket wall which extends in parallel with the strap. In an embodiment, the pocket wall has a wall opening for receiving a housing protrusion at a front face of the housing. At least one of the pocket wall and housing protrusion may be elastically deformable for snapping the housing into the insert pocket. Herewith, the housing is received in a form-fit manner, in particular a snap-fit, in the insert pocket. Advantageously, the form-fit provides a correct orientation of a rear side and front side of the housing, such that the at least one motion sensor inside the housing will get correctly positioned on a lower leg of the user.

In an embodiment of the wearable cueing device according to the invention, the housing protrusion is formed by a button. The button may be a cue activation button for manually activating a cue. A manual activation of a cue may be desired for several reasons. The user may manually activate a cue when he is aware that a gait irregularity is upcoming, or when he knows that a certain motion to be carried out is vulnerable to be disrupted by a gait irregularity. The user may for example manually activate the cue to stand up from a chair and walk away.

In an embodiment of the wearable cueing device according to the invention, the strap comprises a length adjustment buckle and a closure at a substantially fixed position relative to the holder. The closure may for example be formed by a press button or a hook and loop fastener. The closure is formed at the strap ends. In attaching the strap, the user will intuitively place the closure at an outside of the users leg. As a consequence of the fixed position of the holder relative to the closure, the holder will get positioned at a predetermined location at the user's leg. Advantageously, the length adjustment feature in combination with the non-adjustable closure contributes to a precise positioning of the housing, in particular the motion sensor, with respect to the users leg which increases a reliability of the wearable cueing device.

In an embodiment of the wearable cueing device according to the invention, the housing has an upper side to be upwardly directed to obtain the upwards orientation of the housing which is required for a proper operation. The housing may be provided with a mark, e.g. ‘this side up’ to indicate a correct orientation for use. The upper side may be marked with an arrow on an outside of the housing, such that a user understands a proper orientation of the housing. The upper side may be provided with at least one operating button. The operating button at the upper side of the housing contributes to an intuitive correct insertion of the housing into the insert pocket of the strap. The operating button at the upper side of the housing is further beneficial in that the button remains visible and accessible for the user after attachment of the strap to one of the user's legs.

In an embodiment of the wearable cueing device according to the invention, the housing is box-shaped. The housing has a front face, a rear support face to be mated with a user's leg, a left and right side face, and a bottom side. The housing houses electronics which may further contain a power unit, a control unit and a cueing unit. Herewith, the housing may comprise all the electronic components of the wearable cueing device to form a single unit held by the strap. Advantageously, the wearable cueing device is a stand-alone device.

In an embodiment, the housing may comprise an electronic circuit including a transponder to communicate with a remote control unit and a cueing unit, for example implemented by an app on a smartphone or smartwatch.

In an embodiment of the wearable cueing device according to the invention, the cueing unit is an auditory cueing unit for producing auditory cues. In particular, a system formed by a smartwatch operatively connected with the housing can be used as a wearable cueing device in which the smartwatch operates as the auditory cueing unit.

In an embodiment of the wearable cueing device according to the invention, the cueing device further comprises an alarm unit for providing an alarm in case of a fall of the user. The alarm unit may produce an alarm to the user's environment, e.g. a noisy alarm to attract attention of people in a neighbourhood of the user, or a remote alarm as a call for help, e.g. from caregiver or a relative.

Thus, a wearable cueing device is provided comprising a strap with a holder for carrying a housing in a predetermined upwards orientation. The housing is manually attachable to the holder by inserting the housing by a sliding movement into the holder. The holder has an insert pocket which is formed by a carrying loop having an equally sized upper and bottom opening for receiving the housing. To wear the cueing device, the user first attaches the strap to one of the legs, whereafter the user inserts the housing in the predetermined upwards orientation into the holder. The configuration of the strap with holder and housing is beneficial in that an orientation of the strap does not matter for a correct placement of the housing. Herewith, a safety and reliability of the wearable cueing device is improved.

In a first side-aspect of the third aspect of the invention, the cueing device is a wearable cueing device which is formed by a system comprising a combination of a smartwatch and a smartphone, wherein the smartphone functions as a housing provided with at least one motion sensor for providing a sensor signal, and wherein the smartwatch functions as a cueing unit for producing a cue to the user in case of a gait irregularity. The smart phone and the smartwatch are each programmed with an app which is configured to establish a cooperation of the combination of the smart phone and smartwatch to determine and to produce a cue in case of a gait irregularity. In use, the smartwatch is worn on a wrist and the smartphone is positioned at one of the legs of the user. The smart phone may be simply inserted in a hose pocket, such that a strap is not necessary. Alternatively, the smart phone may be attached to the leg of the user by a limb band. The limb band may have a sleeve shaped body made of a stretchable material. By forming the wearable cueing device by commonly available devices, a wearable cueing device is easily available for a person with Parkinson. Beneficially, only a smartwatch app and a smartphone app needs to be downloaded and installed by the user to obtain the wearable cueing device.

In a second side-aspect of the third aspect the invention, the invention relates to a wearable fall prevention device. The fall prevention device is configured to measure a users gait and to determine a gait irregularity which might cause a fall of the user. The fall prevention device comprises a strap which is configured to encircle a user's leg. The strap is provided with a closure for opening and closing the strap. Further, the fall prevention device comprises a housing for housing electronics including at least one motion sensor for providing a sensor signal to a control unit to obtain motion data. The housing has a predetermined upwards orientation for an operational placement relative to the users leg. Beneficially, an attachment of the housing to the users leg in the prescribed upwards orientation may provide accurate measurements of a user's gait to precisely determine an upcoming or occurring gait irregularity. Further, the fall prevention device includes an alarm unit for producing an alarm to the user or other person, like a physician, to trigger attention for an event rendering a fall risk. An alarm signal may be generated to warn a user, and/or an alarm signal may be sent to a remote station, e.g. a relative's smart phone or a caregiver at a remote location.

According to this side-aspect of the invention, an improvement is provided in the manually releasable attachment of the housing to the strap. The holder of the strap is configured to receive the housing in both the predetermined upwards orientation and in an upside down orientation. Herewith, the strap is attachable to one of the user's legs without bothering a correct placement of the housing in the predetermined upwards orientation.

In an embodiment of the fall prevention device, the manually releasable attachment is configured as above described regarding the wearable cueing device. The manually releasable attachment may be configured as an insert pocket having an equally sized upper and lower insertion opening.

In an embodiment of the fall prevention device according to the invention, the housing is attachable to the strap by a first and second press button. The strap has a holder which is formed by a first and second press button member, and the housing includes at a rear side a first and second complementary press button member. In attaching the housing to the strap, the first and second press button define a position of the housing relative to a strap end. In addition, the second press button defines a rotational positioning of the housing.

In an embodiment of the fall prevention device according to the invention, the housing has a housing body including a ‘form follows function’ shape as described above and in the detailed description regarding the wearable cueing device. An arrow shaped housing body is beneficial in providing an intuitive suggestion to the user to hold the housing in the correct predetermined upwards orientation before attaching the housing to the strap.

According to a next side-aspect of the invention, the invention relates to a housing for a wearable device wherein the housing has a housing body including a ‘form follows function’ shape. In particular, a ‘form follows function’ shape as described above and in the detailed description regarding the wearable cueing device. The housing has a predetermined upwards orientation for an operational placement relative to a user's extremity. Beneficially, the shape of the housing intuitively suggests a user to place the housing in a correct orientation before attaching the housing to a strap.

In particular, the wearable device comprises a strap which is configured to encircle a user's extremity, in particular a user's leg. The strap is provided with a closure for opening and closing the strap. Beneficially, an attachment of the housing to the users extremity in the prescribed upwards orientation may provide accurate measurements of a user's gait.

According to this side-aspect of the invention, an improvement is provided in the manually releasable attachment of the housing to the strap. The holder of the strap is configured to receive the housing in both the predetermined upwards orientation and in an upside down orientation. Herewith, the strap is attachable to one of the user's extremity without bothering a subsequent correct placement of the housing in the predetermined upwards orientation.

In an embodiment of the wearable device according to the invention, the housing has a housing body including a ‘form follows function’ shape as described above and in the detailed description regarding the wearable cueing device. An arrow shaped housing body is beneficial in providing an intuitive suggestion to the user to hold the housing in the correct predetermined upwards orientation before attaching the housing to the strap.

In an embodiment of the wearable device, the manually releasable attachment is configured as above described regarding the wearable cueing device. The manually releasable attachment may be configured as an insert pocket having an equally sized upper and lower insertion opening.

In an embodiment of the wearable device according to the invention, the housing is attachable to the strap by a first and second press button, in particular a metal snap button. The strap has a holder which is formed by a first and second press button member, and the housing includes at a rear side a first and second complementary press button member. The first and second press button are positioned in a length or width direction of the strap. In attaching the housing to the strap, the first and second press button define a position of the housing relative to a strap end. In addition, the second press button defines a rotational positioning of the housing.

Embodiments according to the invention are defined by the following clauses:

1. Wearable cueing device (1) for assisting a user during a motion by producing a cue when a gait irregularity is determined, wherein the cueing device (1) comprises:
a strap (3) configured to encircle a user's leg which strap is provided with a closure (30) for opening and closing the strap;
a housing (2) for housing electronics including at least one motion sensor (8) for providing a sensor signal to a control unit (7) to obtain motion data, wherein the housing (2) has a predetermined upwards orientation for an operational placement relative to a users leg;
a cueing unit (6) for producing a cue to the user in case of a gait irregularity;
   wherein the housing (2) is manually releasable connected to the strap (3), wherein the strap comprises a holder (4) forming an insert pocket for carrying the housing, wherein the holder has an insertion upper opening (401) and an insertion lower opening (402) which are equally sized for slidably receiving the housing in the holder.

2. Wearable cueing device according to clause 1, wherein the holder (4) comprises a carrying loop (40) which is preferably made of an elastic material for receiving the housing (2).

3. Wearable cueing device according to any of the preceding clauses, wherein the predetermined upwards orientation of the housing is defined by a form of the housing (2) having a flat upper side (21) and a converging bottom side (26), in particular a rounded bottom side.

4. Wearable cueing device according to any of the preceding clauses, wherein the housing (2) is box-shaped having an upper side (21) to be upwardly oriented, wherein a marker, for example an arrow or a wording ‘this side up’, on the housing indicates a correct orientation of the housing for use.

5. Wearable cueing device according to any of the preceding clauses, wherein the housing (2) is coupled to the holder (4) by a form fit, in particular a snap-fit.

6. Wearable cueing device according to clause 5, wherein a rear side of the housing is provided with a form member (28) and wherein the holder includes a complimentary member (43) which members engage with each other by a form-fit after placement of the housing (2) into the holder (4).

7. Wearable cueing device according to any of the clauses 2-6, wherein the carrying loop (40) forming the insert pocket has a pocket wall (41) extending in parallel with the strap (3), wherein the pocket wall (41) has a wall opening (42) for receiving a housing protrusion (27) positioned at a front side (22) of the housing.

8. Wearable cueing device according to any of the preceding clauses, wherein a front side (22) of the housing (2) is provided with a housing protrusion (27) to be fit into an opening of the holder.

9. Wearable cueing device according to any of the preceding clauses, wherein the strap (3) comprises a length adjustment buckle (33) and wherein the closure (30) has a fixed position at a distance ‘d’ relative to the holder (4), such that the holder gets positioned at a predetermined location at a user's leg, in particular at a peroneus compartment.

10. Wearable cueing device according to any of the preceding clauses, wherein the housing (2) has at least one operating member, e.g. a button (29), at the upper side of the housing.

11. Wearable cueing device according to any of the preceding clauses, wherein the wearable cueing device is a wearable Parkinson cueing device, a so called a Parkinson gait assist device, for producing a cue when determining a gait irregularity relating to Parkinson disease.

12. Wearable cueing device according to any of the preceding clauses, wherein the wearable cueing device comprises an alarm unit for providing an alarm in case of a fall of the user.

### BRIEF DESCRIPTION OF THE DRAWINGS

The invention will be explained in more detail with reference to the appended drawings. The drawings show a practical embodiment according to the invention, which may not be interpreted as limiting the scope of the invention. Specific features may also be considered apart from the shown embodiment and may be taken into account in a broader context as a delimiting feature, not only for the shown embodiment but as a common feature for all embodiments falling within the scope of the appended claims or clauses above, in which:

FIG. 1 shows a wearable cueing device which is attachable by a strap to a lower extremity of a user;

FIG. 2 shows a flow diagram for initiating a cue in three different manners;

FIG. 3 shows the flow diagram of FIG. 2 in which an automatic activation of the cueing unit in case of a FoG detection is further detailed;

FIG. 4 shows a perspective view of the wearable cueing device according to the invention including a housing carried by a holder of a strap;

FIG. 5 shows the strap in an extended view;

FIG. 6 shows a closure of the strap which is normally positioned at a lateral outside of a user's leg;

FIG. 7 shows a length adjustment buckle of the strap;

FIG. 8 shows the holder of the strap in a cross-sectional view;

FIG. 9 shows in a left-sided view different orientations of the strap when respectively attached to a left or right leg of the user, and in a right sided cross-sectional view, a knee angle α between the holder and a central knee position in which the closure is positioned at the lateral outside of the users leg on a user's peroneus longus;

FIGS. 10 and 11 show an intuitive shape of the housing in respectively a front and rear sided perspective view; and

FIG. 12 shows a top view of the housing including switch buttons for switching on or off an auditory or haptic cue.

### DETAILED DESCRIPTION OF THE DISCLOSURE

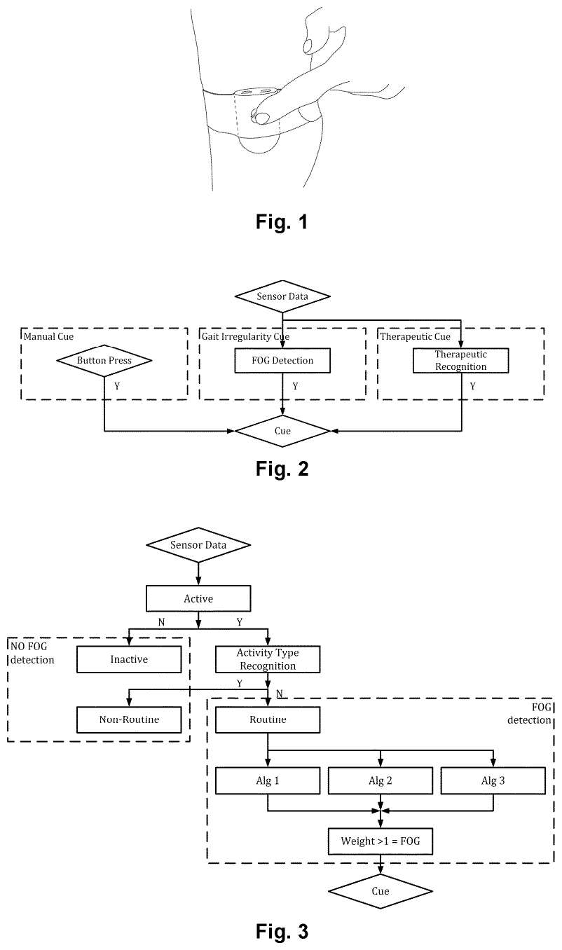

FIG. 1 shows a wearable cueing device which is attachable to a leg of a user. The cueing device includes a housing which is held by a strap. A cueing unit for producing a cue to the user and at least one motion sensor for providing a sensor signal to obtain motion data are housed by the housing. At an outer surface, the housing has a button for self-activation of a cueing unit to produce a cue. By pressing the button, a user has a first possibility to start cueing.

FIG. 2 shows a flow diagram of an operation of the cueing device. Three possibilities are illustrated to activate a cue. Each possibility to start cueing is indicated by dotted lines. A first possibility is a self-activation carried out by the user by pressing a button, a so called manual activation. The flow diagram shows that besides the pressing of the button, two further possibilities to start cueing are arranged.

A gait irregularity can be determined based on sensor data obtained by the at least one motion sensor. Such a gait irregularity may be a detection of a freeze of gait (FoG). Determining a gait irregularity automatically activates the cueing unit. The generated cue supports the user in overcoming an occurring gait irregularity or in preventing an upcoming gait irregularity.

In an embodiment according to a second aspect of the invention, besides a determination of a gait irregularity, based on provided sensor data, a therapeutical user motion may be recognised. The control unit is programmed with a self-activation function based on the therapeutical user motion. The control unit is programmed to recognise the therapeutical user motion when carried out by the user to start cueing. A therapeutical user motion is a user motion which is selected from a group of possible user motions which are proven user motions to overcome or prevent a gait irregularity. The therapeutical user motion may be a preferred user motion selected by the user himself or an assisting physician. Preferably, the therapeutical user motion for cue self-activation is a lower extremity user motion, e.g. a footstamp or a knee bent, which can be accurately measured by the at least one motion sensor. A physician may select a particular user motion which is believed to perform the best for a particular user. Alternatively, a user may select a therapeutical user motion by himself to find out which pre-defined therapeutical user motion performs the best in his situation.

FIG. 3 shows a further elaborated flow diagram of the flow diagram of FIG. 2 in which the automatic cue activation is elaborated in further detail. In FIG. 3, an activation of the cue in case of a determination of a FoG, a FoG detection, is elaborated.

FIG. 3 shows a flow diagram of a cueing device in an embodiment according to the first aspect of the invention. FIG. 3 shows a flow diagram of an embodiment of the computer-implemented method according to the first aspect of the invention. A gait irregularity cue is provided in case of a gait irregularity, in particular a freeze of gait detection, a FoG detection based on positive indications from multiple algorithms.

In a first step of the method, sensor data is obtained. A cueing device provided with the method comprises at least one motion sensor to obtain motion data based on a received sensor signal.

Based on obtained sensor data, it can be established whether or not the user is active. When the user is inactive, no gait irregularity will occur, and the cueing device may remain in a standby status. Based on the sensor data, it can be determined when the user becomes active.

When the user becomes active, in a second step of the method, a routine algorithm is run to determine whether or not an occurring motion of the user is a routine motion. In case that it is determined by the routine algorithm that the occurring motion of the user is a non-routine motion, a risk on an occurrence of a gait irregularity may be neglectable and the cueing device may remain in the standby status. However, in case that it is determined by the routine algorithm that the occurring motion of the user is a routine motion, a gait irregularity might occur and the cueing device is switched to an operational mode in which a single or multiple algorithms are processed to determine a gait irregularity.

Here, the control unit is programmed with a first algorithm, a second algorithm and a third algorithm indicated by Alg1, Alg2 and Alg3. To produce a cue, it is required that at least two outcomes of these algorithms determine a gait irregularity. The control unit is programmed with a weight factor (>1) to indicate an amount of algorithms required to positively determine a gait irregularity before activating a cueing unit. If an amount of positive outcomes is equal or larger than the weight factor, the cue is activated. The weight factor may be adjustable by a user or physician. Alternatively, the weight factor may be dependent on an occurring circumstance or a personal parameter.

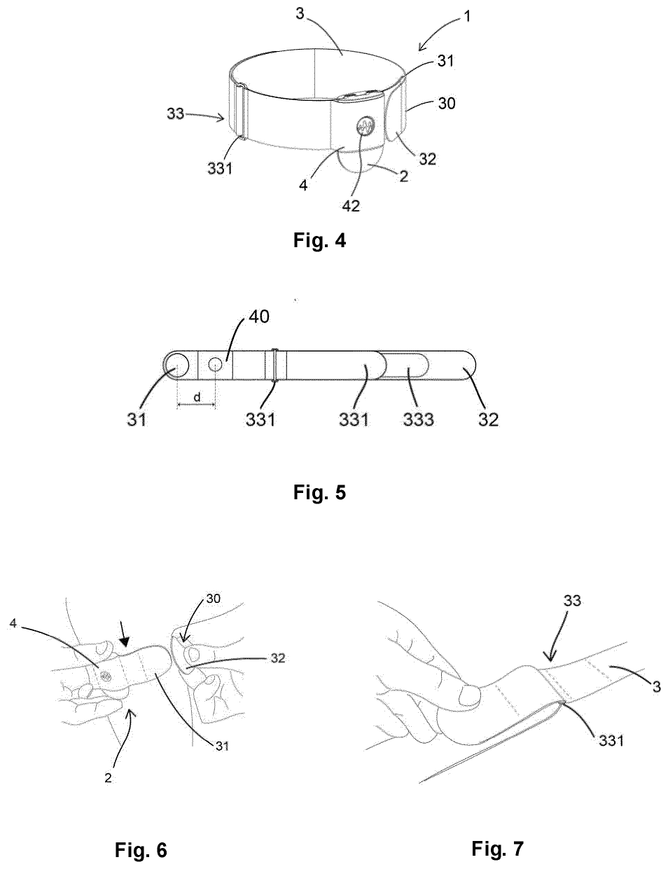

FIG. 4-12 show a wearable cueing device 1 which may include a self-activation function based on a detection of a handsfree predetermined user input and/or which may include multiple algorithms providing multiple outcomes for indicating a gait irregularity. In particular, FIGS. 4-12 show an embodiment of a wearable cueing device according to the third aspect of the invention.

FIG. 4 shows a wearable cueing device 1 in a perspective view. The wearable cueing device 1 is arranged to assist a person during a motion by producing a cue. In particular, the wearable cueing device is a Parkinson gait assist device to guide a person with Parkinson during a gait to prevent or overcome an occurring gait irregularities. A freeze of gait is an example of such a gait irregularity which can be detected as upcoming or occurring when sensing a motion of the user. The wearable cueing device 1 includes a cueing unit 6 for producing a cue to the user in case of a gait irregularity.

The wearable cueing device 1 comprises a strap 3 provided with a holder for 4 holding a housing 2.

FIGS. 5 and 6 show the strap 3. The strap 3 is a knee strap. The strap 3 is configured to be placed around a user's leg at a position just below a knee. The strap 3 is made of a textile material. The strap has a closure 30 to allow a user to manually attach and remove the strap 3 from the leg by opening the closure 30. Here, the closure is made by hook and loop fasteners at both strap ends 31, 32. Further, at an intermediate region, the strap 3 has a length adjustment buckle 33 for adjusting a length of the strap in correspondence with a circumference of a user's leg just below a knee. The length adjustment buckle includes a clip 331. The clip 331 is connected to a first strap portion and configured to receive a second strap portion which is folded back. The first strap portion has a fixed length. The second strap portion is provided with hook and loop fasteners to attach the second strap portion in a back folded position as shown in FIG. 4.

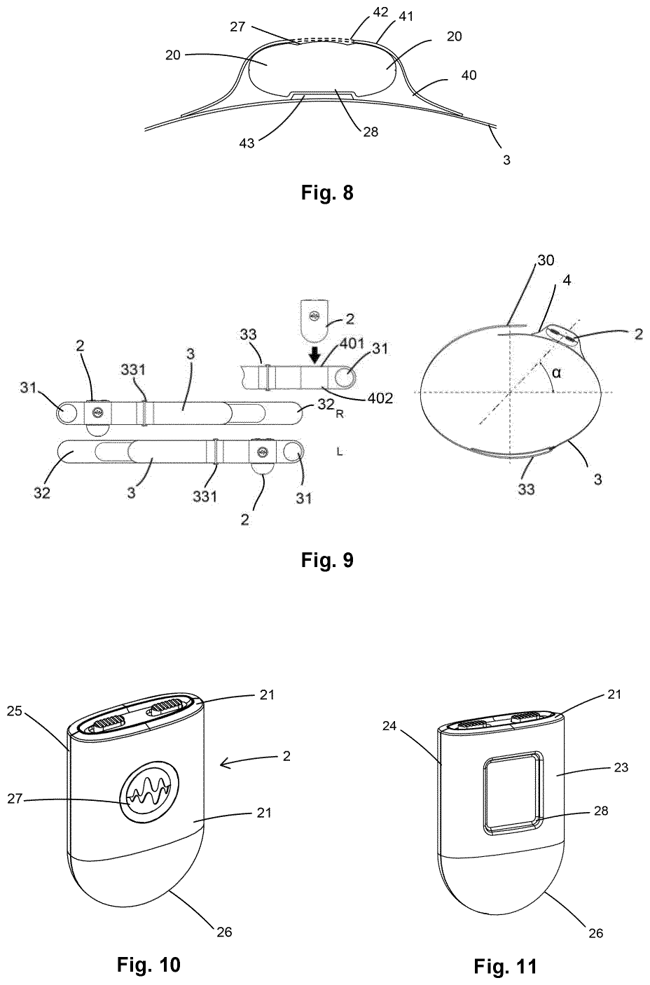

As shown in further detail in FIG. 8, the holder 4 is formed by a carrying loop 40 attached to the strap 3. Here, the carrying loop 40 is made of the same material as the strap 3. The carrying loop 40 has an upper and lower opening 401, 402 which are equally sized. The carrying loop 40 forms an insert pocket for receiving the housing 2. The insert pocket has a pocket wall 41 which has a wall opening 42. The wall opening 42 is configured to receive a housing protrusion 27 at a front side 22 of the housing 2. FIGS. 4 and 10 also show the housing protrusion 27. The housing protrusion 27 is preferably an operating member, in particular a press button, for self-activating a cue.

As further shown in FIGS. 8 and 11, the housing 2 has a housing body 20 which has a form member 28 at a rear side 23. Here, the form member 28 is a housing recess. The form member 28 is configured in correspondence with a complementary member 43 of the holder 4. The form member 28 and the complementary member 43 engage to each other by a form-fit. After insertion of the housing 2 in the insert pocket of the strap, the members 28, 43 contribute to an accurate positioning of the housing 2 relative to a user's leg which improves a reliability of operation.

The housing 2 is attachable to the strap 3 by an inserting motion. The upper and lower opening 401, 402 are equally sized. Hence, the housing 2 is insertable through one of these openings 401, 402 to place the housing 2 behind the carrying loop.

FIG. 9 illustrates in a left sided view a benefit of the configuration of the strap 3. In functioning as a Parkinson gait assist device, in dependence of a user preference, the strap may be attached to a left or right leg of the user. For a proper operation, it is important that the housing 2 is attached to the strap according to the predetermined upwards orientation. To wear the cueing device, the user first attaches the strap 3 to his leg. Intuitively, the user will position the closure 30 of the strap 3 at an outside of his leg. Hence, when placing the strap to the left leg, the strap will get oriented upside down with respect to a placement at the right leg. Advantageously, the configuration of the holder 4 anticipates to these different orientations of a placed strap. The holder 4 has an upper and lower opening 401, 402 which both allow an insertion of the housing 2. Due to the presence of the upper and lower opening 401, 402, the user or a physician can always insert the housing 2 from above (as indicated by an arrow in FIG. 6). Herewith, the configuration of the holder 4 stimulates that the housing 2 will always be oriented in a right manner. The correct placement of the housing 2 increases a reliability of a proper working. Advantageously, the design of the strap 3 and the holder 4 contribute to an increased safety of the Parkinson gait assist device.

In a right-sided view of FIG. 9, a cross-sectional view is shown in which the placement of the housing 2 relative to a user's knee is illustrated. The strap 3 encircles a users leg just below the knee. The closure 30 is positioned laterally outside the users leg which follows from a natural tend of a user.

As also shown in FIG. 5, the holder 4 is positioned at a fixed distance ‘d’ from the first strap end 31 of the closure 30. Herewith, the holder 4 positions an inserted housing 2 at a knee angle α outwards from a central front position at the knee. Preferably, the fixed distance ‘d’ is configured to place the housing 2 onto a peroneus compartment of the user. In particular, the knee angle α is at least 30°, in particular at least 45° which is beneficial to obtain an accurate sensoring by the motion sensor 8 for determining a gait irregularity. Advantageously, the fixed distance ‘d’ between the strap and 31 and the holder 4 contributes to an accurate measurement in determining a gait irregularity. The length adjustment buckle 33 provided separate from the closure 31 and which has a clip 331 attached to the strap portion provided with the holder 4 is beneficial to allow a length adjustment of the strap without changing the fixed distance ‘d’.

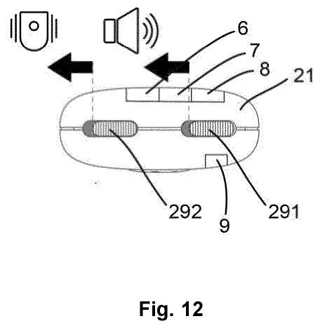

As further shown in FIG. 12, the housing 2 is arranged for housing electronics. The housing has a housing body 20 which houses an electronic circuit including at least one motion sensor 8, e.g. an accelerometer. The wearable cueing device may be a stand-alone device in which the housing further comprises a cueing unit 6, a control unit 7 and a battery 9. Alternatively, the housing 2 of the wearable cueing device 1 may be configured to cooperate with a smart phone or smart watch in which e.g. the cueing unit 6 is provided by the smartphone. For a proper operation of the wearable cueing device, the housing 2 is to be attached to the strap 3 in a predetermined orientation. The predetermined orientation is required to obtain accurate motion data from the at least one motion sensor 8 housed by the housing 2.

As shown in FIGS. 10 and 11, the housing 2 has an upper side 21 which in use is to be held in the upwards direction to obtain accurate measurements of the at least one motion sensor 8. The housing body 20 has a ‘form-follows-function’ configuration. The housing body 20 has a flat upper side 21 and a converging bottom side 26. The bottom side 26 has a rounded or spherical surface. In combination, the flat upper side 21 and the converging bottom side 26 provide an arrow shape configuration of the housing body 20. This arrow shape configuration provides an intuitive suggestion to the user to direct the bottom side 26 downwards and to the upper side upwards. Herewith, the configuration of the housing body 20 contributes to a proper placement of the housing 2 when attaching the housing to the strap 3.

A presence of at least one operating member 29 at the upper side 21, as shown in FIG. 9, may further increase the intuitive suggestion to the user to hold the housing 2 in the correct predetermined orientation before attaching the housing 2 to the strap 3. An operating member 29 may for example be a switch button 291, 292 for switching on/off an auditory or haptic cue.

Thus, the invention provides a wearable cueing device 1 comprising a strap 3 with a holder 4 for carrying a housing 2 in a predetermined upwards orientation. The housing is manually attachable to the holder by inserting the housing by a sliding movement into the holder. The holder has an insert pocket which is formed by a carrying loop having an equally sized upper and bottom opening for receiving the housing. To wear the cueing device 1, the user first attaches the strap 3 to one of the legs, whereafter the user inserts the housing 2 in the predetermined upwards orientation into the holder 4. The configuration of the strap with holder and housing is beneficial in that an upside-down orientation of the strap does not matter for a correct placement of the housing 2. Herewith, a safety and reliability of the wearable cueing device 1 is improved.

Although the present invention has been described in detail, it will be apparent to those skilled in the art that various changes and modifications can be made without departing from the scope of the invention as defined in the clauses and hereinafter claimed. It is intended that all such changes and modifications be encompassed within the scope of the present disclosure, clauses and claims.

Further, it is remarked that any feature of the cueing device according to the invention which is described in the embodiments, defined in a clause and/or mentioned in the dependent claims is in itself considered patentable without any dependency to another presented feature. In particular, any measure presented in a clause or dependent claim is also considered patentable without dependency of the independent claim.

Thus, the invention provides a cueing device for assisting a user during a motion when determining a gait irregularity. The cueing device has a cueing unit for producing a cue, at least one motion sensor for providing a sensor signal to obtain motion data and a control unit operatively connected with the cueing unit and the at least one motion sensor to provide a cue based on a determined gait irregularity. The cueing device has a self-activation function to allow the user to initiate a cue on an own initiative. The self-activation function is based on a hands-free predetermined user input which is detectable by a sensor of the cueing device. The hands-free predetermined user input may be a sound or a specific user motion. Preferably, the user motion is a lower limb motion, for example a toe tapfoot stamp, which can be carried out easily despite of an occurring gait irregularity.

Reference signs list:

| | | | |
|---|---|---|---|
| 1 | cueing device | 33 | length adjustment buckle |
| 2 | housing | 331 | clip |
| 20 | housing body | 4 | holder |
| 21 | upper side | 40 | carrying loop |
| 22 | front side | 401 | upper opening |
| 23 | rear side | 402 | lower opening |
| 24 | left side | 41 | pocket wall |
| 25 | right side | 42 | wall opening |
| 26 | bottom side | 43 | complementary member |
| 27 | housing protrusion / button | ‘d’ | distance holder - closure |
| 28 | form member | ‘α’ | knee angle |
| 29 | operating member | 5 | motion/gait sensor |
| 3 | strap | 6 | cueing unit |
| 30 | closure | 7 | control unit |
| 31 | strap end | 8 | motion sensor |
| 32 | strap end | 9 | battery |

1. Cueing device for producing a cue for assisting a user during a motion when a gait irregularity is determined, wherein the cueing device comprises:
a housing and a strap to wear the cueing device at a user's lower extremity;
a control unit situated in the housing which is programmed by a first algorithm providing a first outcome to determine a gait irregularity, in particular a freeze of gait (FoG), during the motion;
a motion sensor situated in the housing operatively connected to the control unit to obtain motion data based on a received sensor signal; and
a cueing unit operatively connected to the control unit to provide a cue to the user,
wherein the control unit is programmed by at least a second algorithm providing a second outcome to determine a gait irregularity in addition to the first algorithm, wherein the first algorithm and the at least second algorithm are both run on motion data originating from the same motion sensor, wherein a cue is only produced in case that the at least first and second outcomes of the at least first and second algorithms positively indicate for a gait irregularity, wherein the control unit is further programmed by a routine algorithm which is configured to distinguish between a routine or a non-routine motion of the user.

2. The cueing device according to claim 1, wherein the control unit is programmed with at least three different algorithms to provide at least three outcomes to determine a gait irregularity.

3. The cueing device according to claim 1, wherein a cue is only provided if all outcomes of the algorithms positively indicate for a gait irregularity.

4. The cueing device according to claim 1, wherein the required amount of algorithms which have to positively indicate for a gait irregularity before providing a cue is adjustable.

5. The cueing device according to claim 4, wherein at least one algorithm is programmed or manually selectable to be taken into account or cancelled, such that a variety of combinations of algorithms are available.

6. The cueing device according to claim 4, wherein the control unit includes a user interface allowing a person to disable at least one of the available algorithms.

7. The cueing device according to claim 1, wherein at least one algorithm is based on a user profile including at least one user dependent parameter.

8. The cueing device according to claim 7, wherein at least one of the algorithms is distinguished from another algorithm by including a user dependent parameter, for example a BMI or body length, while the other algorithm lacks a user dependent parameter.

9. The cueing device according to claim 1, wherein at least one algorithm includes an environmental parameter, for example a hard or soft floor surface or a shoe type.

10. (canceled)

11. The cueing device according to claim 1, wherein the routine algorithm is carried out before running any of the algorithms to determine a gait irregularity.

12. The cueing device according to claim 11, wherein the control unit is programmed such that if a non-routine motion is determined by the routine algorithm, a running of any of the available gait irregularity algorithms is cancelled.

13. The cueing device according to claim 1, wherein the cueing device is formed by a system including a smart phone and a smart watch which are provided with cooperating apps and in which the smartphone houses the at least one motion sensor to obtain motion data of the user, and in which the smartwatch serves as a cueing unit to produce a cue to the user in case of a gait irregularity.

14. The cueing device according to claim 1, wherein the cue device is a wearable cueing device, wherein the cueing device comprises a strap and a housing for housing the motion sensor which housing is connected to the strap.

15-17. (canceled)

18. A computer-implemented control method for controlling a cueing device configured to provide a cue during a motion of a user, the control method comprising:
receiving a sensor signal from a motion sensor;
acquiring motion data based on the received sensor signal;
running a routine algorithm to distinguish between a routine or non-routine motion of the user
running a first algorithm on the motion data providing a first outcome to determine a gait irregularity;
running at least a second algorithm on the same motion data providing a second outcome to determine a gait irregularity;
providing a cue only if at least two algorithms have both an outcome positively indicating for a determination of a gait irregularity.

19. (canceled)

20. Method for treating a person suffering from a motor disorder of neurological origin by preventing freezing of gait comprising the steps of:
attaching a wearable cueing device at a lower extremity of a person;
receiving a sensor signal from a motion sensor of the cueing device;
acquiring motion data based on the received sensor signal;
running a routine algorithm by a control unit of the cueing device to distinguish between a routine or non-routine motion of the user
running a first algorithm on the motion data by the control unit of the cueing device to provide a first outcome to determine a gait irregularity;
running at least a second algorithm on the same motion data by the control unit of the cueing device to provide a second outcome to determine a gait irregularity;
providing only a cue by a cueing unit of the cueing device if at least two algorithms have both an outcome positively indicating for a determination of a gait irregularity.

21. Method according to claim 20 comprising a step of attaching the cueing device on a predetermined location at a user's leg, optionally at a peroneus compartment, wherein the cueing device comprises:
a housing and a strap to wear the cueing device at a user's lower extremity;
a control unit situated in the housing which is programmed by a first algorithm providing a first outcome to determine a gait irregularity, in particular a freeze of gait (FoG), during the motion;
a motion sensor situated in the housing operatively connected to the control unit to obtain motion data based on a received sensor signal; and
a cueing unit operatively connected to the control unit to provide a cue to the user,
wherein the control unit is programmed by at least a second algorithm providing a second outcome to determine a gait irregularity in addition to the first algorithm, wherein the first algorithm and the at least second algorithm are both run on motion data originating from the same motion sensor, wherein a cue is only produced in case that the at least first and second outcomes of the at least first and second algorithms positively indicate for a gait irregularity, wherein the control unit is further programmed by a routine algorithm which is configured to distinguish between a routine or a non-routine motion of the user.

22. A computer program product comprising a computer readable medium, the computer readable medium having computer readable code embodied therein, the computer readable code being configured such that, on execution by a suitable computer or processor, the computer or processor is caused to perform the method according to claim 21.
